(19) United States
(12) Patent Application Publication
(10) Pub. No.: US 2022/0325316 A1
(43) Pub. Date: Oct. 13, 2022

(54) COMPOSITIONS AND METHODS FOR DETECTING METHYLATED DNA

(71) Applicant: BATTELLE MEMORIAL INSTITUTE, Columbus, OH (US)

(72) Inventor: Rachel R. SPURBECK, Columbus, OH (US)

(21) Appl. No.: 17/633,733

(22) PCT Filed: Aug. 7, 2020
(86) PCT No.: PCT/US2020/045425

§ 371 Date: Feb. 8, 2022

Related U.S. Application Data
(60) Provisional application No. 62/884,942, filed on Aug. 9, 2019.

(51) Int. Cl. C12Q 1/6806; C40B 40/06; C12N 15/10

(52) U.S. Cl. CPC C12Q 1/6806; C40B 40/06; C12N 15/1093

(57) ABSTRACT

Novel methods and compositions are provided for determining global methylation patterns in isolated genomic DNA. The method ustilizes methylation sensitive restriction enzymatic cleavage followed by Next Gernation Sequencing of the remaining DNA to identify sequences comprising methylated nucleic acid residues. In accordance with one embodiment a method is provided for monitoring global methylation patterns in genomic DNAs recovered from organisms or cell populations.

### CROSS REFERENCE TO RELATED APPLICATIONS

This application claims priority under 35 U.S.C. § 119(e) to U.S. Provisional Application Ser. No. 62/884,942 filed on Aug. 9, 2019, the disclosure of which is expressly incorporated herein.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 2.62 kilobytes ACII (Text) file named “298200_ST25.txt,” created on Aug. 7, 2020.

### BACKGROUND

Epigenetics is the study of heritable changes in gene expression that do not involve changes to the underlying primary nucleic acid sequence. Epigenetic change is a regular and natural occurrence but can also be influenced by several factors including age, the environment/lifestyle, and disease state. There is a renewed interest in epigenetics, as epigenic modifications have been associated with a host of disorders including various cancers, mental retardation associated disorders, immune disorders, neuropsychiatric disorders and pediatric disorders.

Epigenetic marks include DNA methylation, histone modifications, and regulatory RNA. Epigenetics is most often studied in multicellular organisms such as humans and plants, where epigenetic marks function in embryogenesis, cellular differentiation, genomic imprinting, and play roles in pathogenesis of diseases such as cancer. In bacteria, histones do not exist; therefore, non-coding RNAs and DNA methylation are the only universal epigenetic marks. The roles of DNA methylation in bacteria range from defense against bacteriophage infection, initiation of DNA replication, DNA repair, and gene regulation. 5N methylcytosine (5mC) is the most common epigenetic mark in higher eukaryotes, whereas 6N methyladenosinde (6 mA) is the most common epigenetic mark in bacteria. However, both marks are present in eukaryotes and prokaryotes, along with other nucleotide base modifications (See FIG. 2). Direct evidence has linked 6 mA to several species of eubacteria, mosquitoes, wheat, protists, and indirect evidence links 6 mA to several archaea and vertebrates. However, methods to determine the global 6 mA patterns using common next generation sequencing (NGS) techniques are not commercially available. Accordingly, the role of 6 mA in eukaryotic genomic function may be under appreciated due to a failure to detect 6 mA.

In prokaryotes, 6 mA not associated with restriction modification systems is produced by the action of two methyl transferases, deoxyadenosine methylase (DAM), which methylates at GATC sites, and DNA methylase N-4/N-6 domain-containing protein (CcrM), which methylates at the motif GANTC. Studies have shown that 6 mA is present in the mitochondrial and chloroplast genomes, and therefore, could play a regulatory role throughout the Tree of Life.

While 5mC can be measured by conventional bisulfite sequencing, global 6 mA patterns at the nucleotide level are currently detected only by PacBio Sequencing (Korlach, J. and S.W. Turner, Curr Opin Struct Biol, 2012. 22(3): p. 251-61), requiring a specialized, costly instrument and significant consumables to reach the depth of sequencing necessary for methylated base calls i.e., 100,000 for a human genome). Thus there is a need for a new methodology that allows for the rapid and cost effective analysis of the presence of 6 mA in a sample of genomic DNA.

In accordance with one embodiment of the present disclosure, applicant provides a novel method, “6 mA-Seq”, to identify 6 mA residues by sequence analysis using an Illumina sequencer or equivalent equipment.

### SUMMARY

Currently, DNA methylation is one of the most broadly studied and well-characterized epigenetic modifications. Bacteria, like eukaryotes, use methylation to regulate gene expression. However, methylation profiling is not common in bacteria due to a lack of methodology pertinent to study of the dominant epigenetic marker in prokaryotes, 6N methyladenine (6 mA). In accordance with one embodiment of the present disclosure a method is provided for assessing global 6 mA profiles in bacterial genomes. The method can be used to monitor changes in methylation patterns of bacterial genomic DNA over time and/or in response to various environmental factors, including for example temperature, availability of nutrients and presence of stimulants or toxins.

In accordance with one embodiment a method is provided for monitoring global methylation patterns in genomic DNAs recovered from organisms or cell populations. More particularly, the method is directed to assessing the methylated state of genomic sequences associated with one or more target restriction enzyme recognition sites. In one embodiment the method of detecting methylated nucleic acid residues comprises the steps of first obtaining a library of genomic DNA and then subjecting the library of genomic DNA to restriction enzymatic digestion with either 1) enzymes that cut only at sites when the DNA is methylated (leaving a library enriched for unmethylated regions) or enzymes that cut only unmethylated regions (leaving a library enriched for methylated sequences). The DNA sequences of the restriction enzyme cleaved library are then analyzed using Next Generation Sequencing (NGS) to determine the sequences remaining in the restriction enzyme cleaved library, wherein comparison of the sequences identified by the Next Generation Sequencing step to a reference library of all available target restriction enzyme recognition sites present in relevant genome reveals those sites that were methylated in the analyzed sample.

In one embodiment the method of detecting methylated nucleic acid residues in a sample comprising genomic DNA comprises the steps of first obtaining a library of genomic DNAs and then contacting the library with a methylation specific restriction enzyme that cleaves said target nucleic acid recognition site only when said target nucleic acid recognition site is unmethylated, to produce a set of digested genomic nucleic acid molecules enriched in methylated sequences. In one embodiment the methylation sensitive restriction enzyme is slected from the restricitons enzymes listed in FIG. 1. In one embodiment the restriction enzyme used to digest the genomic DNA is selected from the group consisting of DpnI, DpnII and MboI.

The DNA sequences of the restriction enzyme cleaved library is then analyzed using Next Generation Sequencing to determine the sequences remaining in the restriction enzyme cleaved library. Comparison of the sequences identified by the Next Generation Sequencing step with a reference genomic sequences (said reference genomic sequence comprising all of the respective genomic sequesce comprising the the restriction enzyme recognistion site) reveals the unmethylated sequence in the analyzed genome as sequences missing relative in the restriction enzyme cleaved library relative to the reference genomic sequences. The sequences detected in the restriction enzyme cleaved library that comprise a target restriction enzyme recognition site are identified as methylated sequences.

In accordance with one embodiment the genomic DNA to be analyzed for the presence of methylated nucleotides (e.g., 6 mA) is processed prior to enzymatic directions. More particularly, a PCR-Free library of genomic DNA is prepared suitable for Next Generation Sequencing. In one embodiment the preparation of the PCR-free library comprises the stesps of isolating genomic DNA from a cell/organism without an amplification step, fragmenting the isolated genomic DNA into DNA sequences less than 1 Kb in length, and typically having an average size of about 300 bp to about 600 bp, and the ligating the fragments of the genomic DNA to adapters that include all necessary components for sequencing primer annealing and attachment to a flow-cell surface for conducting Next-Generation Sequencing.

In one embodiment the step of restriction enzyme digestion of the genomic DNA is conducted using two different restriction enzymes that cleave the same recognition sequence but where one enzyme is sensitive to methylation and the other is not. The initial library of genomic DNA is divided into a first and second pool of genomic DNA wherein the first pool of genomic DNA is digested with a restriction enzyme that cleaves its target nucleic acid recognition site only when said target nucleic acid recognition site is unmethylated to produce a first set of digested genomic nucleic acid molecules (enriched in methylated DNAs), and second pool of genomic DNA is digested with a restriction enzyme that cleaves its target nucleic acid recognition site regardless of the methylated state of the target recognition site to produce a second set of digested genomic nucleic acid molecules. The sequence of the digested nucleic acids of the first and second digested genomic nucleic acid molecules is then determined, typically by using Next Generation Sequencing techinques. The nucleic acid sequences of the first and second digested genomic nucleic acid molecules are compared, and sequences present in the first set of digested genomic nucleic acid molecules relative to the second set of digested genomic nucleic acid molecules are identified as methylated sequences. In one embodiment the restriction enzyme used to digest the first pool of genomic DNA is selected from the group consisting of DpnI, DpnII and MboI and the restriction enzyme used to digest the second pool of genomic DNA is Sau3AI

### BRIEF DESCRIPTION OF THE DRAWINGS

FIG. 1 presents a table of known restriction enzymes that are methylation sensitive. Recognition sites appear in bold type. Nucleotides in addition to the recognition site that are required to produce an overlapping methylation site appears as normal type (not bold). Bases constrained by the requirements of an overlapping methylation site that would otherwise be degenerate (N, R, or Y) are indicated by italics and double underline red. For palindromic enzymes, both ends of the recognition sequence must be considered for possible overlapping methylation, e.g. Clal is blocked by Dam methylation at GATCGAT and ATCGATC.

FIG. 2 presents a listing of the structure of methylated nucleotide bases found in eukarotic and prokaryotic organisms.

### DETAILED DESCRIPTION

### Definitions

In describing and claiming the invention, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment.

As used herein, the term “purified” does not require absolute purity; rather, it is intended as a relative definition. The term “purified nucleic acid” is used herein to describe a nucleic acid which has been separated from other compounds including, but not limited to polypeptides, lipids and carbohydrates.

The term “isolated” requires that the referenced material be removed from its original environment (e.g., the natural environment if it is naturally occurring). For example, a naturally-occurring nucleic acid present in a living animal is not isolated, but the same nucleic acid, separated from some or all of the coexisting materials in the natural system, is isolated.

As used herein the terms “restriction endonuclease” or “restriction enzyme” are used interchangeably and encompass proteins that are able to cleave a double stranded DNA sequence at or near a specific sequence of nucleotides.

As used herein the term “restriction enzyme recognition site” defines locations on a DNA molecule containing a specific sequence of nucleotides that are recognized by the individual restriction enzyme and result in the cleavage of the sequence between two nucleotides within its recognition site, or somewhere nearby.

As used herein the term “methylation sensitive restriction enzyme” encompasses restriction enzymes whose cleavage is blocked or inhibited when the restriction enzyme recognition site is methylated by the cognate methylase.

### EMBODIMENTS

In accordance with one embodiment, the present disclosure is directd to a method for analyzing global methylation patterns in genomes, particularly in bacterial genomes or the genomes of eukaryotic organelles. Restriction enzymes that select for digestion of DNA at sites with differential methylation enable one to remove either unmethylated or methylated DNA and then compare the library that remains to a full genome to identify regions of methylation on a genome wide basis. Such methods can be used to establish correlations between certain methylation patterns and disease states or conditions, and/or determine the impact of various environmental factors on the methylated state of the genome and their corresponding impact on gene expression.

Restriction endonucleases are known that will only cleave their target recognition site when the DNA is in a unmethylated state. Advantageously, as disclosed herein such restriction enzymes can be used to detect the presence of methylated nucleic acids in genomic sequences, and more significantly analyze the methylated state of DNA sequences at genomic level.

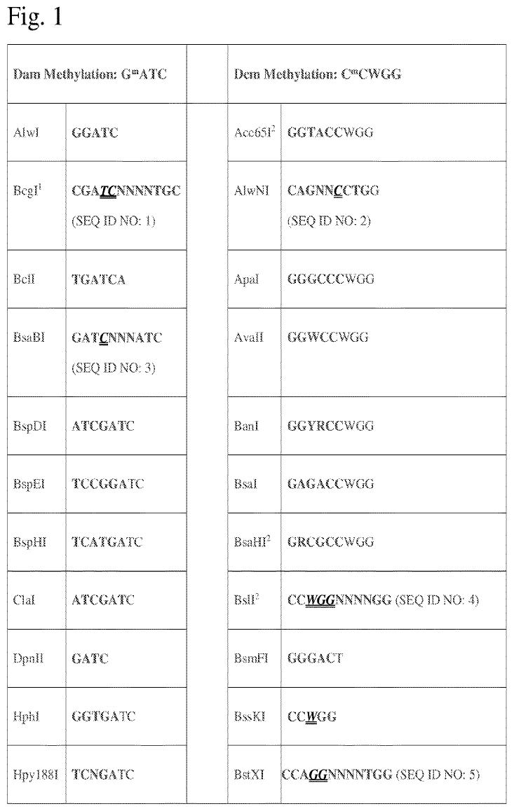

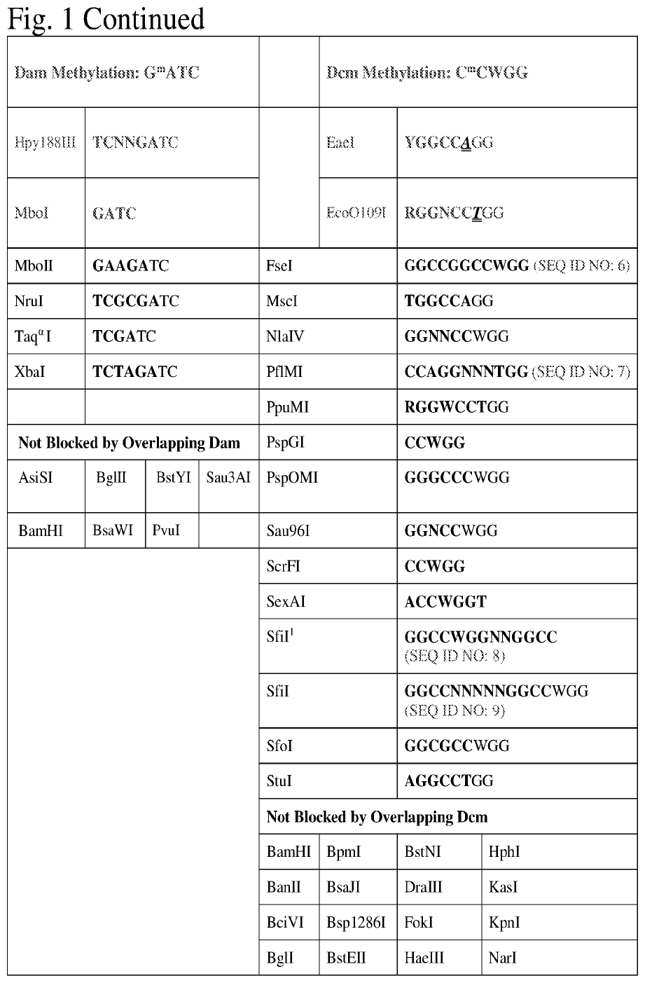

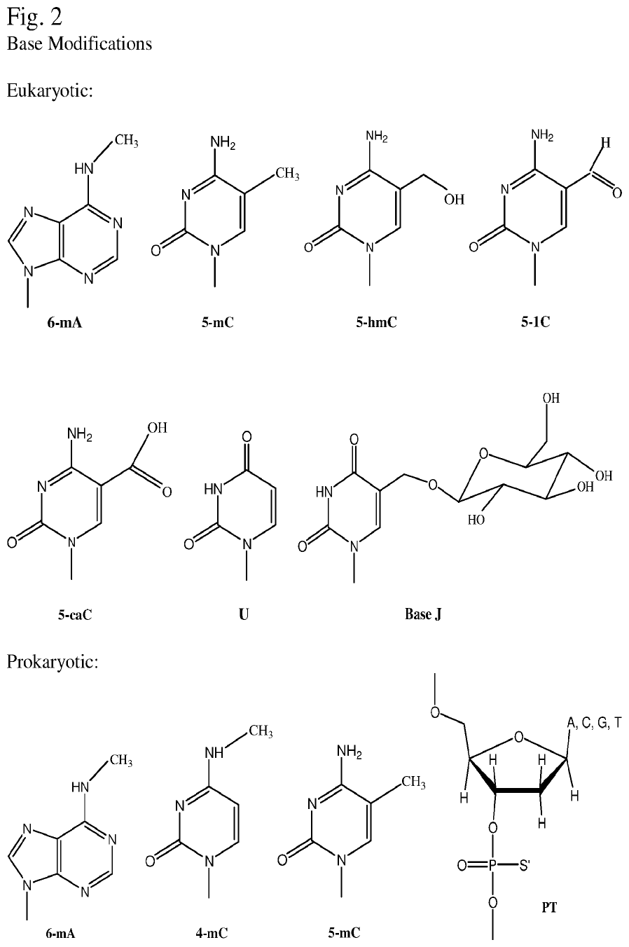

FIG. 1 provides a list of restriction enzymes that are methylation sensitive. Restriction enzyme cleavage is blocked or substantially inhibited when the recognition sequence is methylated by the cognate methylase. More particularly, methylation of nucleic acid bases at or near the restriction enzyme recognition site can block cleavage, leave cleavage unaffected, or slow the rate or extent of cleavage. A restriction enzyme database, REBASE, is known to those skilld in the art for providing more detailed information regarding methylation sensitive restriction enzymes.

In accordance with the present disclosure, methylation sensitive restriction enzymes can be used to determine and monitor methylation patterns of genomic DNAs on a global level. In particular, one can track how methylation of genomic DNA sequences is altered by exposure to various environmental factors or genetic background. The methods disclosed herein can be used to analyze genomic DNA isolated from any cell, including organelle genomic DNA from chloroplasts and/or mitochondria of eukaryotice cells. In one embodiment the methylation state of genomic DNA isolated from bacterial cells is analyzed using the methods disclosed herein.

There are three common DNA methylases that are present in bacterial cells such as E. coli: the Dam methylase, Dcm methylase and EcoKI methylase. The methylase encoded by the dam gene (Dam methylase) transfers a methyl group from S-adenosylmethionine (SAM) to the N6 position of the adenine residues in the sequence GATC. The Dcm methylase (encoded by the dcm gene; referred to as the Mec methylase in earlier references) methylates the internal (second) cytosine residues in the sequences CCAGG and CCTGG at the C5 position. The EcoKI methylase, M. EcoKI, modifies adenine residues in the sequences AAC(N6)GTGC and GCAC(N6)GTT. EcoKI sites (˜1 site per 8 kb) are much less common than Dam sites (˜1 site per 256 bp) or Dcm sites (˜1 site per 512 bp) in DNA of random sequence (GC=AT).

In one embodiment genomic DNA is isolated from a cell and subjected to enyzymatic digestion using a methylation sensitive endonuclease. In one embodiment the isolated genomic DNA is first processed and a library of the genomic DNA is prepared from the processed genomic DNA. In accordance with one embodiment, the genomic DNA is first fractionated to reduce the average size of the genomic DNA prior to preparation of the library. The genomic DNA can be fractionated using any standard technique known to those skilled in the art to reduce the size of the average genomic DNA to less than 1 Kb in length. In one embodiment the DNA is fractionated by ultasonification and/or nebulization, including for example the use of the Covaris Adaptive Focused Acoustics (AFA) technology (Covaris, Inc., 14 Gill Street, Unit H, Woburn, Mass., 01801-1721 USA) to generate fragments having an average size of about 350 bp to about 550 bp, or about 250 bp to about 350 bp. Ultasonification shearing can be used to generate double-stranded DNA (dsDNA) fragments with 3′ or 5′ overhangs (see U.S. Pat. No. 9,103,755).

The fractionated DNA is then linked to the appropriate adapters to create a library of factionated genomic DNA sequences. Preferably the creation of the genomic libraries is conducted in the absence of a PCR amplification step (i.e., a PCR-free library). In accordance with one embodiment the fragmented genomic DNA is subjected to a repair step wherein the overhangs resulting from fragmentation are convereted into blunt ends. In one embodiment a 3′ to 5′ exonuclease activity removes the 3′ overhangs, and a 5′ to 3′ polymerase activity completes the 5′ overhangs. In a final step the fragments of genomic DNA are ligated to adapters that include all necessary components for sequencing primer annealing and attachment to a flow-cell surface for conducting Next-Generation Sequencing. See Kozarewa et al, Nature Methods 2009; 6:291-295; and Illumina TruSeq DNA PCR-Free Illumina, Inc. 5200 Illumina Way, San Diego, Calif. 92122 USA).

In accordance with one embodiment PCR-free genomic libraries are prepared from the cells whose genomic DNA will be assessed for methylation patterns. PCR amplification is commonly used in generating libraries for Next-Generation Sequencing (NGS) to efficiently enrich and amplify sequenceable DNA fragments. However, it introduces bias in the representation of the original complex template DNA. Accordingly, in one embodiment of the present invention libraries of genomic DNA will be prepared in the absence of a PCR amplification step and the digestion with methylation sensitive restriction enzymes will be conducted on the PCR-free library components.

In one embodiment a method is provided for analyzing 6 mA methylation patterns in genomic DNA, including genomic DNA isolated from prokaryotics. The method comprises obtaining a PCR-Free library of genomic sequences and subjecting the library to at least one restriction enzyme digestion (cleaving DNA but only when the recognition site is unmethylated (e.g., lacking a 6 mA residue). Optionally a second enzymatic digestion can be conducted wherein the second enzyme cleaves the same sites as the first but irregardless of whether the site is methylated or unmethylated. The digested library can optionally be amplified by PCR to enrich for uncut sequences. The enzymatic diegested library sequence can be sequenced (typically by NGS) and compared to the corresponding reference sequences (comprising all known target recognition sites for the relevant genome) to determine where methylation was or was not, depending on the enzyme used. In one embodiment this analysis is conducted on bacterial DNA or other DNA to assess N6-methyladenosine patterns over time and/or and in relation to exposure to different environmental factors.

Bacteria primarily use 6 mA for epigenetic regulation of gene expression. In accordance with one embodiment methods are provided for detecting 6 mA in genomic DNA using Illumina platforms. In one embodiment, whole genome sequencing libraries will be prepared, polymerase chain reaction-free, from DNA from each organism or cell line. These libraries are treated with a mix of enzymes that remove library molecules that contain methylated adenosines (such as 6 mA), leaving behind only molecules that do not have a methylated adenosine. The libraries will then be amplified to enrich the 6 mA libraries for complete molecules and sequenced on the appropriate Illumina sequencing platform. Adapters and low-quality reads will be trimmed with Trimmomatic software prior to analysis of the data. Data analysis includes alignment to the reference sequence by BWA-MEM software and visualization through IGV genome viewer. A bed file of each genome will be produced with the coordinates of possible adenosine methylation sites. Coverage at these possible methylation sites will be determined and compared to the average coverage across the genome. Regions where reads are absent or more than two standard deviations below the mean in coverage represent regions where adenosine bases were not methylated. Analysis of methylated versus unmethylated sites in this manner will provide a global map of 6 mA across the genome.

A list of methylation sensitive and insensitive restriction enzymes is provided in FIG. 1. In accordance with one embodiment the methylation sensitive restriction enzyme used in the present invention is selected from the group consisting of DpnI, DpnII and MboI. MboI, DpnI and DpnII each recognize and cleave at the recognition sequence GATC and both enzymes are blocked by dam methylation, but not blocked by dcm methylation. Hinfl recognizes and cleaves at the recognition sequence GANTC, and cleavage is not blocked by either dam methylation, or dcm methylation. HpaII and Mspl recognize and cleave at the recognition sequence CCGG recognition site and can be used for C5-methylcytosine detection.

The global analysis of 6 mA in species beyond prokaryotes will revolutionize the field of epigenetics, demonstrating the impact of 6 mA in response to environmental changes. State-of-the-art analysis methods overlook 6 mA, although it is known to regulate processes in both prokaryotes and eukaryotes. It is now apparent that 6 mA is an important regulator in all organisms but requires a lower limit of detection than possible back in the 1970s. The 6 mA modification can regulate gene expression in specific organelles, including mitochondria which is targeted by environmental contaminants such as PFOA and causes a large impact on the lives of multicellular eukaryotes.

In accordance with one embodiment a kit is provided for analyzing DNA for the presence of 6 mA. The kit comprises one or more methylation sensitive restriction enzymes, optionally selected from the group consisting of DpnI, DpnII and MboI. The kit may contain additional reagents for preparing PCR-free libraries.

In accordance with embodiment 1, a method of detecting methylated nucleic acid residues, optionally a N6-methyladenosine (6 mA) modification, in one or more target nucleic acid recognition sites present in a sample of genomic DNA is provided, wherein said method comprising the steps of

a) obtaining a library of genomic DNA;

b) contacting said library with a methylation specific restriction enzyme that cleaves said target nucleic acid recognition site only when said target nucleic acid recognition site is unmethylated to produce a set of digested genomic nucleic acid molecules; and

c) analyzing the digested genomic nucleic acid molecules using next generation sequencing to determine the methylation state of said target nucleic acid recognition sites.

In accordance with embodiment 2 the library of genomic DNA of embodiment 1 is prepared by isolating genomic DNA from a cell without a PCR amplification step; fragmenting the genomic DNA to an average size of about 300 bp to about 600 bp; ligating the fragmented genomic DNA to adapters wherein said adapter comprise a primer sequence for sequence analysis and optionally additional sequences complementary to sequences linked to a solid support.

In accordance with embodiment 3, the method of embodiment 1 or 2 is provided wherein the library of genomic DNA is contacted with two or more methylation sensitive restriction enzymes.

In accordance with embodiment 4, the method of any one of embodiments 1 to 3 is provided wherein the genomic DNA is isolated from a prokaryote.

In accordance with embodiment 5, the method of any one of embodiments 1 to 4 is provided wherein the analyzing step comprises determining the nucleic acid sequence of said digested genomic nucleic acid molecules and comparing those nucleic acid sequences to a reference set of nucleic acids that represent all available target nucleic acid recognition sites present in said sample of genomic DNA, wherein sequences missing from the digested genomic nucleic acid molecules relative to said reference set represents a unmethylated target sequence in said sample of genomic DNA.

In accordance with embodiment 6, a method of detecting methylated nucleic acid residues, optionally a N6-methyladenosine (6 mA) modification, in one or more target nucleic acid recognition sites present in genomic DNA is provided wherein, said method comprising the steps of
a) obtaining a library of genomic DNA fragments, wherein said genomic DNA fragments comprise isolated genomic DNAs that have been fragmented to have an average size of about 300 bp to about 600 bp, and have been further modified by covalent linkage of an adapter sequences that comprises a DNA sequencing primer to each DNA fragment, optionally wherein the libraray consists of genomic DNA fragments that have not been subjected to PCR;
b) contacting said library with a methylation specific restriction enzyme that cleaves said target nucleic acid recognition site, optionally wherein the target site comprises the sequence of GATC, only when said target nucleic acid recognition site is unmethylated to produce a set of digested genomic nucleic acid molecules; and
c) analyzing the digested genomic nucleic acid molecules using next generation sequencing to determine the methylation state of said target nucleic acid recognition sites.

In accordance with embodiment 7, the method of embodiment 6 is provided wherein the methylation specific restriction enzyme is selected from the group consisting of DpnI, DpnII and MboI.

In accordance with embodiment 8, the method of embodiment 6 or 7 is provided wherein the genomic DNA fragments further comprise sequences complementary to sequences linked to a solid support.

In accordance with embodiment 9, the method of any one of embodiments 6-8 is provided wherein the library of genomic DNA is contacted with two or more methylation sensitive restriction enzymes.

In accordance with embodiment 10, the method of any one of embodiments 6-9 is provided wherein the genomic DNA is isolated from a prokaryote.

In accordance with embodiment 11, the method of any one of embodiments 6-9 is provided wherein the analyzing step comprises determining the nucleic acid sequence of said digested genomic nucleic acid molecules and comparing those nucleic acid sequences to a reference set of nucleic acids that represent all available target nucleic acid recognition sites present in said sample of genomic DNA, wherein sequences missing from the digested genomic nucleic acid molecules relative to said reference set represents a unmethylated target sequence in said sample of genomic DNA.

In accordance with embodiment 12, the method of any one of embodiments 6-11 is provided wherein the library of genomic DNA of step a) is divided into a first and second pool of genomic DNA, and said method comprises the steps of
contacting said first pool of genomic DNA with first restriction enzyme that cleaves said target nucleic acid recognition site only when said target nucleic acid recognition site is unmethylated, to produce a first set of digested genomic nucleic acid molecules;
contacting said second pool of genomic DNA with a second restriction enzyme that cleaves said target nucleic acid recognition site in the presence or absence methylation, to produce a second set of digested genomic nucleic acid molecules, with the proviso that the first and second restriction enzymes each have the same nucleic acid recognition site;
determining the nucleic acid sequence of the first and second digested genomic nucleic acid molecules using Next Generation Sequencing;
comparing the nucleic acid sequnces of the first digested genomic nucleic acid molecules to the nucleic acid sequnces of second digested genomic nucleic acid molecules; and
identifying nucleic acid sequences present in the first digested genomic nucleic acid molecules that are missing in the second digested genomic nucleic acid molecules as methylated sequences.

In accordance with embodiment 13, the method of any one of embodiments 6-12 is provided wherein the target nucleic acid recognition site comprises a nucleic acid sequence of GATC or GANTC, optionally wherein the target nucleic acid recognition site comprises a nucleic acid sequence of GATC.

In accordance with embodiment 14, a method of detecting genomic sequences comprising N6-methyladenosine (6 mA) is provided wherein said method comprises the steps of

a) obtaining a library of genomic DNA fragments, wherein said genomic DNA fragments comprise isolated genomic DNAs that have not been subjected to PCR, have been fragmented to have an average size of about 300 bp to about 600 bp and have been further modified by covalent linkage of an adapter sequences that comprises a DNA sequencing primer to each DNA fragment;

b) contacting the genomic DNA fragments of said library with a methylation sensitive restriction enzyme that cannot cleave its recognition site when 6 mA is present in the restriction site or within 1 or 2 nucleotides of said recognition site to produce a set of digested genomic nucleic acid molecules;

c) obtaining the sequence of the digested genomic nucleic acid molecules; and

d) analyzing the sequence data generated in step c) to identify sequences as being unmethylated when the sequence is not detected relative to a reference library of sequences known to be present in said genome and comprising the recognition site of said methylation sensitive restriction enzyme.

In accordance with embodiment 15, the method of embodiments 14 is provided wherein the genomic DNA of said library is contacted with two or more methylation sensitive restriction enzymes.

In accordance with embodiment 16, the method of any one of embodiments 14 or 15 is provided wherein the methylation sensitive restriction enzyme is selected from the group consisting of MboI, DpnI and DpnII.

In accordance with embodiment 17, the method of any one of embodiments 14-16 is provided wherein the genomic DNA is prokaryotic DNA.

In accordance with embodiment 18, the method of any one of embodiments 14-17 is provided wherein the library of genomic DNA fragments is prepared by

isolating genomic DNA from prokaryotic cells without a PCR amplification step;

fragmenting the genomic DNA to an average size of about 300 bp to about 600 bp;

ligating the fragmented genomic DNA to adapters wherein said adapters comprise a primer sequence for sequence analysis and optionally additional sequences complementary to sequences linked to a solid support.

In accordance with embodiment 18, the method of any one of embodiments 14-17 is provided wherein the library of genomic DNA of step a) is divided into a first and second pool of genomic DNA, and said method comprises the steps of
contacting the first pool of genomic DNA with a first restriction enzyme that cannot cleave its recognition site when 6 mA is present in the restriction site or within 1 or 2 nucleotides of said recognition site, to produce a first set of digested genomic nucleic acid molecules;
contacting the second pool of genomic DNA with a second restriction enzyme that cleaves said target nucleic acid recognition site in the presence or absence methylation, to produce a second set of digested genomic nucleic acid molecules, with the proviso that the first and second restriction enzymes each have the same nucleic acid recognition site; and
determining the nucleic acid sequence of the first and second digested genomic nucleic acid molecules using Next Generation Sequencing; and
comparing the nucleic acid sequnces of the first digested genomic nucleic acid molecules to the nucleic acid sequnces of second digested genomic nucleic acid molecules; and
identifying nucleic acid sequences present in the first digested genomic nucleic acid molecules that are missing in the second digested genomic nucleic acid molecules as sequences containing 6 mA residues.

In accordance with embodiment 19, the method of any one of embodiments 14-18 is provided wherein the methylation sensitive restriction enzyme is selected from the group consisting of DpnI, DpnII and MboI.

In accordance with embodiment 20, the method of any one of embodiments 18-19 is provided wherein the first restriction enzyme is selected from the group consisting of DpnI, DpnII and MboI and the second restriction enzyme is Sau3AI.

### Example 1

### Identification of 6 mA in Genomic Sequences

A proof of concept study was conducted using two strains of Escherichia coli. One strain was derived from E. coli K-12 with the genotype dam⁻/dcm⁻, causing it to be unable to methylate adenosine or cytosine. The other strain was E. coli ATCC 25922, which is wild-type dam⁺/dem⁺, enabling it to methylate its genome at both cytosine and adenosine. PCR-free Illumina sequencing libraries were prepared from DNA from both strains. These libraries were then treated with enzymes that remove library sequences that contained unmethylated adenosines leaving behind only sequences that contained potential 6 mA sites. The dam⁻/dcm⁻ strain had 19146 possible 6 mA sites of which none were methylated according to the 6 mA sequencing analysis, whereas the dam⁺/dcm⁺ strain had 98.8% of its 20460 possible 6 mA sites methylated (Table 1). This data demonstrates the utility of the 6 mA-Seq method for detection of differential 6 mA.

TABLE 1
6mA sequencing coverage statistics for dam/dcm and
dam+/dcm+ E. coli strains

| | # of | | Coverage at | |
|---|---|---|---|---|
| dam/dcm | possible | Total genome | possible | |
| (+/-) | 6mA sites | coverage | 6mA sites | % methylated |
| -/- | 19146 | 61.8 | 0 | 0% |
| -/- | 19146 | 10.7 | 0 | 0% |
| +/+ | 20460 | 49.3 | 46 | 98.8% |
| +/+ | 20460 | 16.5 | 16 | 98.8% |

This method does not require any specialized equipment, utilizes a protocol that is very similar to whole genome sequencing on the Illumina platform, and costs the same as whole genome sequencing. Therefore, the method is easy to use by any lab with access to an Illumina sequencer. The current method should be expandable to detect differential 6 mA methylomes across the Tree of Life as the enzymes used to target 6 mA will work on any 6 mA methylated DNA with the same efficiency.

### Impact of Differential Culture Conditions on Global Methylation

Two Escherichia coli strains, E coli ATCC 25922 with both dam and dcm activity and E coli K 12 with genotype dam−/dcm− were cultured in different media. DNA from each strain was extracted for further analysis using methylation specific restriction enzymes and next generation sequencing. Data analysis consisted in evaluating methylation patterns to find unmethylated locations as absence of sequencing reads (See Table 2).

218 GATC sites demonstrated hyper or hypomethylation in E. coli ATCC25922. 3 GATC loci had consistent hypomethylation in all four growth conditions (position start: 777328, 3928044, and 5032297), whereas 2 were consistently hypermethylated (position start: 2907398 and 2907498). 9 GATC sites (position start: 1661619, 2581408, 2581441, 2581801, 2582072, 2582134, 2718715, 2906872, and 2907142) were consistently hypermethylated in M9 minimal media regardless of carbon source and were not in LB. 9 GATC sites were hypomethylated only in LB (position start: 409277, 479355, 1579192, 1684645, 2181531, 2498588, 2957634, 3782914, and 4031299). LB shared 6 hypomethylated GATC sites with M9-glycerol and M9-glucose (position start: 665850, 786885, 3782985, 3928100, 5031607, and 5032236).

The data demonstrates the utility and high sensitivity of 6 mA Seq for detection of differentially methylated loci in bacteria. While this method is limited by the identification of methylation at the restriction enzyme motifs, the analysis can be expanded by the use of additional methylation sensitive enzymes to digest the library of genomic sequences, thus analyzing methylation at motifs beyond GATC, which will increase the diversity of epigenetic signatures that can be identified by this technique.

As demonstrated 6 mA Seq analytical technique readily identifies sites with differential methylation patterns. E coli which cannot methylate adenine has no coverage at GATC sites, whereas E coli with DAM methylase has 98.8% of sites methylated

6 mA hyper and hypomethylation status changes in different growth conditions at loci across the genome.

LB grown E coli cultures are very different in methylation pattern from those in M9 minimal media, with only 5 GATC sites consistently hyper or hypomethylated between all conditions tested. The 3 hypomethylated sites correspond to a biosynthetic threonine ammonia lyase, a tRNA Ala and a location approximately 100 bp from this tRNA Ala. The two hypermethylated sites correspond to IS 3 family transposase and ClbS/DfsB family four helix bundle protein.

In each culture condition there were unique E coli hyper or hypomethylated sites compared to other conditions tested.

TABLE 2
Differentially methylated GATC sites by culture media

| | # Hypermethylated | # Hypomethylated |
|---|---|---|
| Culture Media | GATCs (% Unique) | GATCs (% Unique) |
| LB | 2 (0%) | 25 (36%) |
| M9-sorbitol | 73 (64%) | 7 (43%) |
| M9-glucose | 88 (63%) | 39 (59%) |
| M9-glycerol | 41 (51%) | 22 (27%) |
| LB or M9-sorbitol | 0 | 0 |
| LB or M9-glucose | 0 | 4 |
| LB or M9-glycerol | 0 | 3 |
| M9-sorbitol or M9-glucose | 14 | 0 |
| M9-sorbitol or M9-glycerol | 1 | 1 |
| M9-glucose or M9-glycerol | 8 | 3 |
| LB, M9-glycose, or M9-glycerol | 0 | 6 |
| M9 independent of carbon source | 9 | 0 |
| All | 2 | 3 |

1. A method of detecting methylated nucleic acid residues in one or more target nucleic acid recognition sites present in genomic DNA, said method comprising the steps of
a) obtaining a library of genomic DNA fragments, wherein said genomic DNA fragments comprise isolated genomic DNAs that have not been subjected to PCR, have been fragmented to have an average size of about 300 bp to about 600 bp and have been further modified by covalent linkage of an adapter sequences to each DNA fragment wherein the adapter sequence comprises a DNA sequencing primer;
b) contacting said library with a methylation specific restriction enzyme that cleaves said target nucleic acid recognition site only when said target nucleic acid recognition site is unmethylated to produce a set of digested genomic nucleic acid molecules; and
c) analyzing the digested genomic nucleic acid molecules using next generation sequencing to determine the methylation state of said target nucleic acid recognition sites.

2. The method of claim 1 wherein the methylation specific restriction enzyme is selected from the group consisting of DpnI, DpnII and MboI.

3. The method of claim 2 wherein said genomic DNA fragments further comprise sequences complementary to sequences linked to a solid support.

4. The method of claim 1 wherein the library of genomic DNA is contacted with two or more methylation sensitive restriction enzymes.

5. The method of claim 4 wherein the genomic DNA is isolated from a prokaryote.

6. The method of claim 1 wherein said analyzing step comprises determining the nucleic acid sequence of said digested genomic nucleic acid molecules and comparing those nucleic acid sequences to a reference set of nucleic acids that represent all available target nucleic acid recognition sites present in said sample of genomic DNA, wherein sequences missing from the digested genomic nucleic acid molecules relative to said reference set represents a unmethylated target sequence in said sample of genomic DNA.

7. The method of claim 1 wherein the library of genomic DNA of step a) is divided into a first and second pool of genomic DNA, and said method comprises the steps of
contacting said first pool of genomic DNA with first restriction enzyme that cleaves said target nucleic acid recognition site only when said target nucleic acid recognition site is unmethylated, to produce a first set of digested genomic nucleic acid molecules;
contacting said second pool of genomic DNA with a second restriction enzyme that cleaves said target nucleic acid recognition site in the presence or absence methylation, to produce a second set of digested genomic nucleic acid molecules, with the proviso that the first and second restriction enzymes each have the same nucleic acid recognition site;
determining the nucleic acid sequence of the first and second digested genomic nucleic acid molecules using Next Generation Sequencing;
comparing the nucleic acid sequences of the first digested genomic nucleic acid molecules to the nucleic acid sequences of second digested genomic nucleic acid molecules; and
identifying nucleic acid sequences present in the first digested genomic nucleic acid molecules that are missing in the second digested genomic nucleic acid molecules as methylated sequences.

8. The method of claim 7 wherein the target nucleic acid recognition site comprises a nucleic acid sequence of GATC or GANTC.

9. The method of claim 7 or 8, wherein the first restriction enzyme is selected from the group consisting of DpnI, DpnII and MboI and the second restriction enzyme is Sau3AI.

10. A method of detecting genomic sequences comprising N6-methyladenosine (6 mA) within a target nucleic acid recognition site or within 1 or 2 nucleotides of said target nucleic acid recognition site, said method comprising the steps of
a) obtaining a library of genomic DNA fragments, wherein said genomic DNA fragments comprise isolated genomic DNAs that have not been subjected to PCR, have been fragmented to have an average size of about 300 bp to about 600 bp and have been further modified by covalent linkage of an adapter sequences to each DNA fragment wherein the adapter sequence comprises a DNA sequencing primer;
b) contacting the genomic DNA fragments of said library with a methylation sensitive restriction enzyme that cannot cleave said target nucleic acid recognition site when 6 mA is present in the restriction site or within 1 or 2 nucleotides of said recognition site to produce a set of digested genomic nucleic acid molecules;
c) obtaining the sequence of the digested genomic nucleic acid molecules; and
d) analyzing the sequence data generated in step c) to identify sequences as being unmethylated when the sequence is not detected relative to a reference library of sequences known to be present in said genome and comprising the recognition site of said methylation sensitive restriction enzyme.

11. The method of claim 10 wherein the genomic DNA of said library is contacted with two or more methylation sensitive restriction enzymes.

12. The method of claim 10 wherein the methylation sensitive restriction enzyme is selected from the group consisting of MboI, DpnI and DpnII.

13. The method of claim 10 wherein said genomic DNA is prokaryotic DNA.

14. The method of claim 10 wherein the library of genomic DNA fragments is prepared by
isolating genomic DNA from prokaryotic cells without a PCR amplification step;
fragmenting the genomic DNA to an average size of about 300 bp to about 600 bp;
ligating the fragmented genomic DNA to adapters wherein said adapters comprise a primer sequence for sequence analysis and optionally additional sequences complementary to sequences linked to a solid support.

15. The method of claim 10 wherein the library of genomic DNA of step a) is divided into a first and second pool of genomic DNA, and said method comprises the steps of
contacting the first pool of genomic DNA with a first restriction enzyme that cannot cleave said target nucleic acid recognition site when 6 mA is present in the restriction site or within 1 or 2 nucleotides of said recognition site, to produce a first set of digested genomic nucleic acid molecules;
contacting the second pool of genomic DNA with a second restriction enzyme that cleaves said target nucleic acid recognition site in the presence or absence methylation, to produce a second set of digested genomic nucleic acid molecules, with the proviso that the first and second restriction enzymes each have the same nucleic acid recognition site; and
determining the nucleic acid sequence of the first and second digested genomic nucleic acid molecules using Next Generation Sequencing; and
comparing the nucleic acid sequences of the first digested genomic nucleic acid molecules to the nucleic acid sequences of second digested genomic nucleic acid molecules; and
identifying nucleic acid sequences present in the first digested genomic nucleic acid molecules that are missing in the second digested genomic nucleic acid molecules as sequences containing 6 mA residues.

16. The method of claim 15 wherein the methylation sensitive restriction enzyme is selected from the group consisting of DpnI, DpnII and MboI.

17. The method of claim 15 wherein the first restriction enzyme is selected from the group consisting of DpnI, DpnII and MboI and the second restriction enzyme is Sau3AI.


SEQUENCE LISTING (9 sequences)

SEQ ID NO: 1 - DNA, 12 bp, Artificial Sequence
  Restriction site target sequence
  misc_feature (6)..(9): n is a, c, g, or t
cgatcnnnntgc

SEQ ID NO: 2 - DNA, 10 bp, Artificial Sequence
  Restriction site target sequence
  misc_feature (4)..(5): n is a, c, g, or t
cagnncctgg

SEQ ID NO: 3 - DNA, 10 bp, Artificial Sequence
  Restriction site target sequence
  misc_feature (5)..(7): n is a, c, g, or t
gatcnnnatc

SEQ ID NO: 4 - DNA, 11 bp, Artificial Sequence
  Restriction site target sequence
  misc_feature (6)..(9): n is a, c, g, or t
ccwggnnnngg

SEQ ID NO: 5 - DNA, 12 bp, Artificial Sequence
  Restriction site target sequence
  misc_feature (6)..(9): n is a, c, g, or t
ccaggnnnntgg

SEQ ID NO: 6 - DNA, 11 bp, Artificial Sequence
  Restriction site target sequence
ggccggccwgg

SEQ ID NO: 7 - DNA, 11 bp, Artificial Sequence
  Restriction site target sequence
  misc_feature (6)..(8): n is a, c, g, or t
ccaggnnntgg

SEQ ID NO: 8 - DNA, 13 bp, Artificial Sequence
  Restriction site target sequence
  misc_feature (8)..(9): n is a, c, g, or t
ggccwggnnggcc

SEQ ID NO: 9 - DNA, 16 bp, Artificial Sequence
  Restriction site target sequence
  misc_feature (5)..(9): n is a, c, g, or t
ggccnnnnnggccwgg